(19) United States
(12) Patent Application Publication
(10) Pub. No.: US 2012/0022038 A1
(43) Pub. Date: Jan. 26, 2012

(54) ANIMAL TREATMENT FORMULATION AND METHODS OF USE

(75) Inventors: Wayne Frederick Leech, Auckland (NZ); Fadil Al Alawi, Auckland (NZ); Kiro Risto Petrovski, Palmerston North (NZ)

(73) Assignee: MASTITIS RESEARCH CENTRE LIMITED, AUCKLAND (NZ)

(21) Appl. No.: 13/131,861

(22) PCT Filed: Nov. 25, 2009
(86) PCT No.: PCT/NZ09/00255

§ 371 Date: Oct. 14, 2011

(30) Foreign Application Priority Data
Nov. 28, 2008 (NZ) 573297

(51) Int. Cl. A61K 31/545; A61P 31/04

(52) U.S. Cl. 514/197

(57) ABSTRACT

The present invention relates to a formulation for administration to the teat canal of the mammary gland of an animal, the formulation including: a physical barrier material, characterised in that the formulation includes sufficient antibiotic in relation to physical barrier material such that the formulation is configured to disintegrate over a period of time after administration to the teat canal.

### TECHNICAL FIELD

This invention relates to an animal treatment formulation and methods of use.

More specifically, this invention relates to a formulation for the treatment or prevention of mammary gland infections such as mastitis.

### BACKGROUND ART

One of the major problems associated with dairy cows is the high occurrence of bovine mastitis, and in particular new infections occurring during the dry or drying off period when dairy cows are susceptible to mastitis. This problem also applies to other lactating animals, but for the purposes of this discussion, reference is made to bovine mastitis.

The present invention has particular application to heifers. Heifers have not previously calved and require different treatment from other cows.

For example, the teat canals in heifers only open for the first time part way into the pre-calving period. This means that they cannot or do not need to receive treatment at the early part of the dry period.

Further, heifers appear to be even more susceptible to mastitis once their teats start leaking prior to their first calving.

Finally, heifers are unused to being milked or handled and therefore most treatment processes can cause heifers far more stress than other cows.

The dominant pathogens associated with dry period mastitis infections include Streptococcus uberis, Streptococcus dysgalactiae and Staphylococcus aureus.

A number of treatments have previously been developed to overcome the occurrence of mastitis, these include DCT (Dry Cow Therapy), external teat seals and internal teat seals. However, as discussed below, there are many disadvantages of these treatments, particularly with regard to their use on heifers.

A common method of treating bovine mastitis is with intramammary antibiotics.

DCT is a method that was used to prevent new intramammary infections occurring in dry cows. This is the treatment of all cows at the start of the dry off period (healthy as well as mastitis-positive) with antibiotic therapy by an infusion of all udder quarters with a long acting antibiotic. This treatment is intended cover the dry period. This has a number of disadvantages.

One disadvantage is that the antibiotic often does not remain at therapeutic levels for the entire dry period. This can lead to increased susceptibility to mastitis when the level drops, buildup of resistance amongst the microorganisms, or the need for further administration.

Further, this infusion cannot be used on heifers because the teat canals in heifers have not yet opened at the start of pregnancy period. If the same infusion was introduced to the heifers at a later period when their teat canals are open, there maybe an overload of antibiotic which could extend into the milking period.

It should be appreciated that when cows are treated with antibiotics, antibiotic residues can be present in the milk. As a result of this, milk needs to be withheld for a period of time following antibiotic administration.

The need to withhold milk is an inconvenience to the farmer. In order to overcome this, the farmer has to either ensure that the milk is diverted away from the holding tank through separate milk lines, or depending on the system, keep the treated animals separate and milk these at a separate time.

The loss of milk, and the time required to implement withholding periods after treatment with antibiotics can result in significant economic losses—unless handled correctly.

Thus, it can be seen that heifers ought not to be treated with the same DCT as used on other cows.

To overcome general problems with DCT, teat seals both external and internal were developed to provide a physical barrier to prevent microorganisms from gaining access in to the teat or udder. Again, both these have significant disadvantages.

External teat seals provide a physical barrier across the entrance to the teat canal. The end of each teat is dipped in an acrylic, latex or polymer-based external teat sealant after milking is finished—at the start of the dry period or for heifers in the late pre-calving period.

Once applied, the external teat seal dries to generate a film that prevents the entry of bacteria into the teat canal.

Unfortunately, the external teat seals are not robust and persistency is often less than six days. This has lead to a recommendation that the seals are applied not only at the start of the dry period, but are also reapplied during this period. This therefore increases the time required by the farmer to check and retreat the cows multiple times during the dry period as well as the cost of doing so.

The use of internal teat seals is another way of trying to prevent mastitis infection during the dry period.

Internal teat seals are thick pastes that are infused into each quarter of the cow's udder at drying off. The internal teat seal forms a physical barrier within the teat canal, thereby preventing micro-organisms from accessing the teat canal.

Many currently available internal teat seals including a heavy metal salt such as bismuth sub-nitrate mixed into a gel base. The gel base may by polyethylene gel, which solidifies after administration.

The gel base may also include a vehicle such as liquid paraffin. For example as in New Zealand Patent No. 336153.

A significant disadvantage of using internal teat seal alone is that it is unable to act against micro organisms which are already present in the teat, or which may be introduced during administration of the teat seal.

In some instances, such as when an animal is known to be infected, antibiotic treatment might be applied along with, or prior to administration of the internal teat seal.

New Zealand Patent No. 258199 relates to a veterinary composition containing the antibiotic cloxacillin in aqueous suspension including a suspension aid, a buffer and a surfactant.

The composition also includes a heavy metal salt and gel base which acts as a physical teat seal. This patent discloses the use of two independent compositions that are administered at the same time. The intramammary composition including cloxacillin is administered first, then followed by the teat seal.

A disadvantage of this prior art product is that microbes which may already be present in the teat are sealed inside and may be become infectious. In addition, the animal's natural sealing process may also be disrupted, slowed or otherwise altered, which may cause harm to the animal.

Another problem with internal teat seals which are bismuth-based are that they are almost too stable. After calving farmers are required to manually strip each sealed teat of this teat seal material. This process is time consuming, but also with regard to heifers can be very distressing. Heifers are unused to milking sheds and having their udder handled. Therefore, heifers tend to be flighty, shy away and kick out at farmers tempting to strip their teats. This is obviously unsatisfactory and potentially dangerous.

Further, existing internal teat sealants require very high hygiene at administration which is often impractical. In addition, it is preferred that technicians apply this product.

It should be appreciated that a product which prevents new infection by pathogenic micro organisms during the late pre-calving period and also avoids altering natural processes would be advantageous.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided a formulation for administration to the teat canal of the mammary gland of an animal, the formulation including:

a physical barrier material,
characterised in that
the formulation includes sufficient antibiotic in relation to physical barrier material such that the formulation is configured to disintegrate over a period of time after administration to the teat canal.

According to a further aspect of the present invention there is provided a use of a formulation as substantially described above, in the manufacture of the medicament to prevent or ameliorate mastitis.

The term ‘infection’ or grammatical variations thereof refers to the invasion of an animal's body by micro-organisms.

The term ‘prevent’ or grammatical variations thereof refers to keeping, averting or hindering an infection from occurring.

The term ‘mammary gland’ or grammatical variations thereof refers to the whole of the udder of an animal, including the secreting tissue, connective tissue, glands and teats.

The term ‘teat canal’ or grammatical variations thereof refers to the teat canal or aperture from the teat cistern, where the milk is extracted from.

The term ‘antibiotic’ refers to a drug that treats infections internally within the body.

The term ‘gelling compound’ or grammatical variations thereof refers to compounds that promote coagulation or thickening of one or more further compounds together.

The term ‘dry period’ or grammatical variations thereof refers to the period where milking for the lactation season has ceased as the cows have no milk to expel and are therefore ‘dry’. A person skilled in the art will understand that this time period may commence is early to mid summer until spring, when the cows calve.

The term ‘late pre-calving period’ or grammatical variations thereof refers to the period where udders start forming secretion pre-calving. A person skilled in the art will understand that this time may commence 2-4 weeks prior to calving.

The term ‘coherent’ or grammatical variations thereof refers to something that is sticky in that it binds to itself and to other material.

The term ‘temporary’ or grammatical variations thereof refers to the physical barrier as not lasting or not being permanent.

Preferably, the physical barrier may disintegrate to leave minimal residue at calving. Preferably, the physical barrier naturally deteriorates or degrades, without the need for physical removal that is required for the prior art formulations and seals.

The inventors of the present invention have unexpectedly found that by including a sufficient amount of antibiotic such as penicillin into a teat seal formulation, the physical barrier provided is temporary in nature and naturally dissipates or disintegrates over time.

This provides an advantage over the prior art, as the formulation of the present invention does not need to be removed or stripped from the teat canal after calving, unlike a number of the teat seals of the prior art.

Additionally, the inventors of the present invention also noted that the formulation of the present invention substantially enhanced the animal's natural teat sealing process or keratin production in the teat canal. The inventors' findings showed that there was a greater quality of the natural keratin production in the animals that had the formulation of the present invention applied, in comparison to animals who did not receive any treatment.

Further, the inclusion of antibiotic within the teat seal means that any infections present within the teat canal at the time of administration are killed off while the teat is sealed. This overcomes one of the problems associated with the prior art in that infections could grow within a sealed teat if they are already present at the time of administration.

Plus, having the antibiotic within the formulation enables an easy one-step administration—unlike the prior art.

Another advantage of including antibiotic is that once the teat seal disintegrates, there may be a small residual effect (which dissipates before calving) preventing new infections from occurring.

The inventor has found that the inclusion of an antibiotic in combination with a barrier material that disintegrates over a predefined period of time with the teat canal can ensure that the antibiotic is introduced to the animal at the required dosage rate.

The upper and lower ranges of the concentration of antibiotic required in the teat seal to be effective in the animal over the various dissolution periods depends on the antibiotic used. The applicant has trialled Cephalonium at a concentration of 250 mg in 4 gm product/teat, while for Penicillin (Procaine and/or Benzathin Penicillin) it varied from 0.8 to 1 gm in 4 gm product/teat

It should be appreciated that the use of antibiotics in an animal should be approached with considerable care.

If antibiotics are introduced into the animal at a low concentration, then it is likely to encourage the development of antibiotic resistant micro-organisms. Likewise, if the antibiotic is only delivered to the animal for a short period of time antibiotic resistant micro-organisms are may also develop.

If the antibiotic is delivered for a long period of time at a high concentration and is absorbed into systemic distribution, then off-target tissues or parts of the animal may be affected, such as the gastrointestinal tract. Further, a high level of antibiotic within the animal can lead to the need to withhold milk for a period of time as the antibiotic may be present and therefore excreted in the milk.

In a preferred embodiment there will be a single dosage of 0.2 to 2 grams per quarter, preferably 0.2-0.5 g depending on the antibiotic (cephalosporins are less 0.2-0.5, while penicillins can go up to 1 gram per quarter). The antibiotic should be dispersed into the teat canal and cistern and not be absorbed into the mammary tissue or bloodstream to any great degree.

The concentration of the antibiotic released from the formulation per day must be sufficient to ensure that the concentration of antibiotic within the teat canal and cistern is above the minimum inhibitory concentrations for the common mastitis pathogens.

As can be appreciated, the amount of antibiotic included also affects the actual disintegration rate of the teat seal which is an important component of the present invention. This has taken some trial and experimentation to determine not only the appropriate amount of antibiotic to treat mastitis without a withholding period, but also the appropriate ratio to barrier material to ensure that the appropriate release rate and ultimate disintegration of the teat seal occurs.

The table below is indicative of approximate release rates found for antibiotics with a physical barrier material such as barium sulphate or bismuth sub-nitrate.

TABLE A

| | Barium sulphate OR | |
|---|---|---|
| | Bismuth subnitrate | |
| Antibiotic (% w/w) | (% w/w) | Release rate |
| 10% (0.5 g antibiotic/syringe) | 60 | 8-12 weeks |
| 20% (1 g antibiotic/syringe) | 50 | 7-10 weeks |
| 30% (1.5 g antibiotic/syringe) | 40 | 4-7 weeks |
| 40% (2 g antibiotic/syringe) | 30 | 2-4 weeks |
| >50% | Nil | 1-5 days |

It can be seen from this table that the introduction of antibiotic at a greater amount than 50% can lead to a very small release rate, say in the order of 1 to 5 days which is insufficient to provide the protection required. The ideal release rate with the present invention is in the order of 8-12 weeks which means the amount of antibiotic required is at the lower end of the scale.

Preferably, the formulation, prior to use, is a paste. Preferably, the paste is administered through the teat canal, for example using a tube or syringe. Once administered, the formulation solidifies in the teat canal to form a substantially solid and coherent temporary physical barrier.

Preferably, the physical barrier may have sufficient coherence to allow it to flex with movement of the sides of the teat canal and/or lower portion of teat cistern. It will be appreciated that this increases the seal created by the physical barrier of the formulation and the side of the teat canal and/or lower portion of teat cistern, to decrease any gap or channel between the seal and the teat canal and/or lower portion of teat cistern.

The formulation of the present invention may be used to prevent and/or ameliorate mastitis. Preferably, the formulation may be used to prevent and/or ameliorate mastitis in dairy animals, preferably the formulation may be administered to sheep, goats or cows, preferably dairy cows.

Preferably, the formulation of the present invention is administered at substantially the start of an animal's dry period, during the drying off period or in the late pre-calving period prior to first calving in primiparous heifers.

It should be appreciated that heifers or prima gravida animals have different requirements to older animals. Usually, their teats are not open until just a few weeks before calving. At that stage, the teats may start to leak and open up and this is where infections are most likely to occur. It should also be appreciated that the younger animals are not accustomed to being handled and therefore requires a formulation that the administration and removal thereof is as stress free as possible.

Preferably, the physical barrier include's barium based compound which may be a barium salt. In one preferred embodiment, the barium salt may be barium sulphate. Barium sulphate is advantageous in that it is cheaper and more environmentally friendly compound than previously used compounds, such as bismuth sub-nitrate.

Preferably, the barium based compound is micronised.

In a preferred embodiment the barium sulphate is present in the formulation at a concentration of approximately 40% to 85% w/w. More preferably, the barium sulphate may be present in the formulation at a concentration of approximately 67% w/w.

It is envisaged that the barium sulphate particles settle into the teat canal or/and lower portion of teat cistern, contribute to substantially sealing off the teat canal or/and lower portion of teat cistern with the temporary physical barrier.

It has been found that barium based physical barrier material does not require stripping from the teats—unlike bismuth based material. However, it should be appreciated that the physical barrier can include any physiologically suitable material which provides the sealing function required.

As an example, bismuth sub nitrate could be used.

The formulation may include a carrier. The carrier may be a gel or an oil or a combination of both a gel and oil, the gel may act to increase the viscosity of the oil, on administration through the teat canal.

Preferably, the gelling compound or compounds are present at a concentration within the range of approximately 1 to 12% w/w of the oil component. More preferably, the gel compound or compounds may be present at a concentration of approximately 5.5 to 6.0% w/w of the oil compound.

Preferably, the gelling compound is aluminium stearate. One skilled in the art would be aware that any other suitable gelling compounds could also be used in conjunction with the present invention. Preferably, the aluminium stearate may be present at a concentration of approximately 1.8% w/w of the final product.

In embodiments where the carrier includes at least one oil, the oil is preferably selected as being an oil that may be difficult for the animal to metabolise. More preferably, the oil may be non-absorbable. More preferably, the oil may act as a vehicle.

Preferably, the oil may be present at a concentration of approximately 30% w/w. It will be appreciated that the concentration may vary depending on the other components used, and may be varied to provide the desired characteristics of the formulation. Therefore, more preferably the oil may be at a concentration determined by the calculation of: 100% minus the other components to be included.

Preferably, the oil may be liquid paraffin. However, this should not be seen as limiting, as one skilled in the art would be aware that a range of different oils may be utilised, for example in some instances vegetable or another mineral oil may be utilised.

The right particle size of the barium sulphate (micronised powder) is very important for structure of the product and hence its viscosity and syringability.

At present there is no strict ratio between the barium sulphate or Bi subnitrate to the oil. In a preferred embodiment it is 60:30 barium sulphate:heavy mineral oil and 60 (Bi subnitrate):31 heavy mineral oil.

A preferred ratio of antibiotic:barium sulphate OR bismuth subnitrate:heavy mineral oil is 10:60:30 To 40:30:30

However if a treatment substance such as an antibiotic is added to the formulation, this ratio would be different. It could, for example, be 62:28 barium sulphate:Cepha DR™ (when Cephalonium is added) or could, for example, be 39-43:34 barium sulphate:Lactapen HDR™ (when Procaine and/or Benzathin Penicillin is added).

It will be appreciated that the preferred physical characteristics of the formulation of the present invention may be provided by the barium sulphate, along with at least one oil and a gelling agent.

In some further preferred embodiments, the formulation may also include other additives to provide the required consistency, physical properties and/or behaviour. For example, the formulation may include a thickening agent, such as Aerosil 200, and/or preservatives, such as methylparaben (as free acid) and/or propylparaben (as free acid).

The applicants consider the rheology of the formulation is important, this includes such features as the ability to flow under high shear forces, being sufficiently fluid that once administered it spreads to form a reasonable seal with the side of the teat canal or/and lower portion of teat cistern and at least some elastic properties to allow the formulation to be able to move and flex with movement of the teat canal or/and lower portion of teat cistern.

It should be appreciated, the need to withhold milk is an inconvenience to the farmer. In order to overcome this the farmer has to either ensure that the milk is diverted away from the holding tank through separate milk lines, or depending on their systems, keep the treated animal separate and milk the animal at a separate time.

The loss of milk and the time required to implement withholding period after treatment with antibiotics, can result in significant economic losses.

However, antibiotics are highly effective at treating mastitis—if the appropriate antibiotic dosage and timing of such dosage is provided.

The use of the present invention addresses a number of these concerns.

The dissolution rate of the barrier material can be made such that the appropriately high dosage level of antibiotic is provided within the teat canal and cistern. Once the teat seal has totally disintegrated, there is no further supply of antibiotic—which means that the period of time over which the antibiotic can be delivered can be determined by the dissolution rate of the teat seal. This means that if a teat seal with a known dissolution rate is placed into a heifer at the appropriate time, the antibiotic can have been processed prior to the mandatory withholding period of 8 milkings has elapsed.

This action of the antibiotic and the teat seal is complemented by the discovery of the inventor that a natural teat seal in the form of the keratin plug still forms while the artificial teat seal is in position.

Thus, it can be seen that the initial introduction of the artificial teat seal prevents new micro-organisms from entering the teat canal. The antibiotic is then released at the required dissolution rate to kill off existing micro-organisms. Once the antibiotic has dissipated, the natural teat seal provides a physical barrier preventing further entry of the micro-organisms into the canal.

As the artificial teat seal has dissolved, there is no need to strip it out. The natural teat seal comes out naturally around calving.

### BRIEF DESCRIPTION OF DRAWINGS

Further aspects of the present invention will become apparent from the following description which is given by way of example only and with reference to the accompanying drawings in which:

FIG. 1 represents average values of product present in the teats at different time points.

FIG. 2 illustrates changes in scores amongst front teats of the same cow at each time point, and

FIG. 3 represents changes of scores amongst rear teats of the same cow at the same time point, and

FIGS. 4-6 illustrate x-rays comparing dissolution of teats within udders.

### BEST MODES FOR CARRYING OUT THE INVENTION

Below are examples illustrating suitable formulations for use with the present invention. It should be noted that the amount of antibiotic to physical barrier material is one that ensures the release rate in the desired range, that is in the order of 8-12 weeks while ensuring the appropriate immunological level.

### Example 1

| | | | qty for |
|---|---|---|---|
| No | Ingredients | 102 gms | 0.7 kgs |
| 1 | Penicillin V* | 10 | 70.28 |
| 2 | Bismuth subnitrate | 60 | 420 |
| 3 | Aluminium stearate | 1.5 | 10.5 |
| 4 | Methyl paraben | 0.04 | 0.28 |
| 5 | Propyl paraben | 0.02 | 0.14 |
| 6 | Heavy liquid paraffin | 30.44 | 213.08 |
| | Total | 102 | 714 |

### Example 2

| | | | qty for |
|---|---|---|---|
| No | Ingredients | 102 gms | 0.7 kgs |
| 1 | Penicillin V* | 20 | 140.56 |
| 2 | Bismuth subnitrate | 50 | 350 |
| 3 | Aluminium stearate | 1.5 | 10.5 |
| 4 | Methyl paraben | 0.04 | 0.28 |
| 5 | Propyl paraben | 0.02 | 0.14 |
| 6 | Heavy liquid paraffin | 30.44 | 213.08 |
| | Total | 102 | 714 |

### Example 3

| | | | qty for |
|---|---|---|---|
| No | Ingredients | 102 gms | 0.7 kgs |
| 1 | Penicillin V* | 30 | 210.84 |
| 2 | Bismuth subnitrate | 40 | 280 |
| 3 | Aluminium stearate | 1.5 | 10.5 |
| 4 | Methyl paraben | 0.04 | 0.28 |
| 5 | Propyl paraben | 0.02 | 0.14 |
| 6 | Heavy liquid paraffin | 30.44 | 213.08 |
| | Total | 102 | 714 |

### Example 4

| | | | qty for |
|---|---|---|---|
| No | Ingredients | 102 gms | 0.7 kgs |
| 1 | Penicillin V* | 40 | 281.12 |
| 2 | Bismuth subnitrate | 30 | 210 |
| 3 | Aluminium stearate | 1.5 | 10.5 |
| 4 | Methyl paraben | 0.04 | 0.28 |
| 5 | Propyl paraben | 0.02 | 0.14 |
| 6 | Heavy liquid paraffin | 30.44 | 213.08 |
| | Total | 102 | 714 |

A discussion of one way the present invention can be implemented follows:

Cows were x-rayed in the insemination race and cattle crush. The teats on the left side were repeatedly x-rayed at six time points (conducted on 31^{st} Jan., 4th, 7th, 15^{th} 22^{nd} Feb. and 7 Mar. 2008) for most of the enrolled cows. Two additional exposures of whole udders were taken at each time point.

There was no evidence of any leakage of the product from the teats.

### Preliminary Results Analysis:

The presence or absence of the product, in the teats of the left side form the enrolled cows, was subjectively judged based on the scoring scale presented in Table 1.

TABLE 1
Scoring scale for presence or absence of product in the teat cavity

| | |
|---|---|
| 0 | None Seen |
| 1 | Very Small Amount |
| 2 | Small Amount |
| 3 | Moderate Amount |
| 4 | High Amount |
| 5 | Nearly All or ALL |

The results from different x-raying time points are presented in Table 2. The values for the udder at the first time point were all zero and were omitted (no dispersion yet) in Table 2 and the analysis. On the 15^{th} Feb. 2008, the product was not visible (suspected dispersed in small particles, not visible by naked eye on x-ray film) and these values were also omitted from Table 2 and the analysis.

TABLE 2
Scores of the presence or absence of product per cow quarter at different time points

| Cow | | 31^{st} January | 4^{th} February | 8^{th} February | 15^{th} February | 22nd February | 7th March |
|---|---|---|---|---|---|---|---|
| ID | Rx¹ | Front | Front | Front | Front | Front | Front |
| 39 | 1 | 5 | 2.5 | 1 | 0.01 | 0.01 | 0 |
| 46 | 1 | 5 | 1.5 | 0.25 | 0.01 | 0 | 0 |
| 48 | 2 | NA | NA | NA | NA | .² | NA |
| 55 | 1 | 5 | . | 2 | 0.25 | 0 | 0 |
| 69 | 1 | 5 | 3.5 | 3.5 | 0 | . | 0 |
| 81 | 2 | 0 | NA | NA | NA | . | NA |
| 82 | 1 | 5 | 2 | 0.01 | 0.03 | . | 0 |
| 100 | 1 | 5 | 3.5 | 0.05 | 0 | . | 0 |
| 132 | 1 | 5 | 3.5 | 0.01 | 0 | . | 0 |
| 140 | 1 | 5 | 2 | 2 | 0.25 | 0.01 | 0 |
| 186 | 1 | 5 | 3.5 | 0 | 0 | 0 | 0 |
| 189 | 1 | . | 1.5 | 1 | 0.01 | 0.25 | 0 |

| | | | | | | |
|---|---|---|---|---|---|---|
| Average | 4.091 | 2.611 | 0.982 | 0.056 | 0.045 | 0 |

¹Treatment group;
²missing value

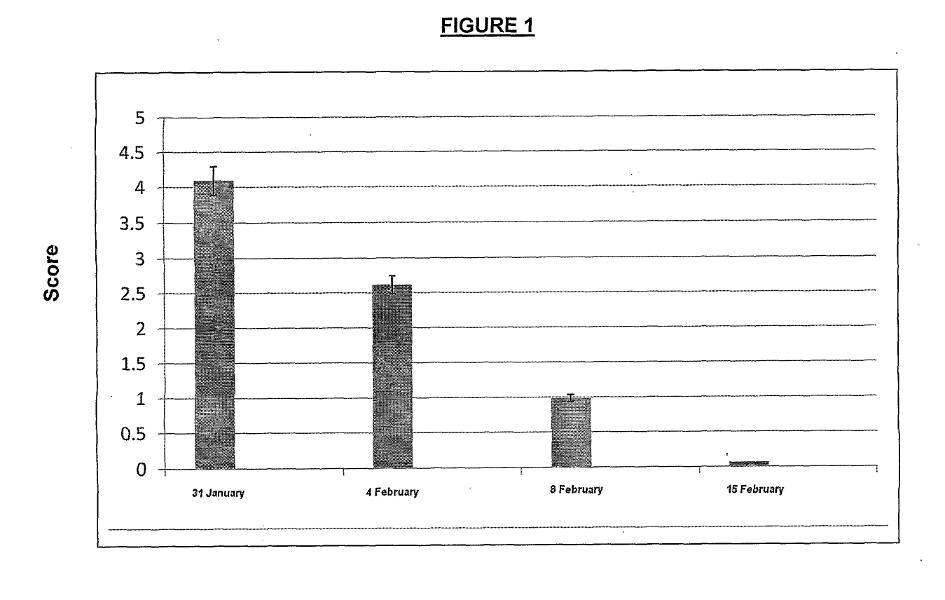

Averages of scores of product presence in the teats per group and their 95% Confidence interval are presented in FIG. 1.

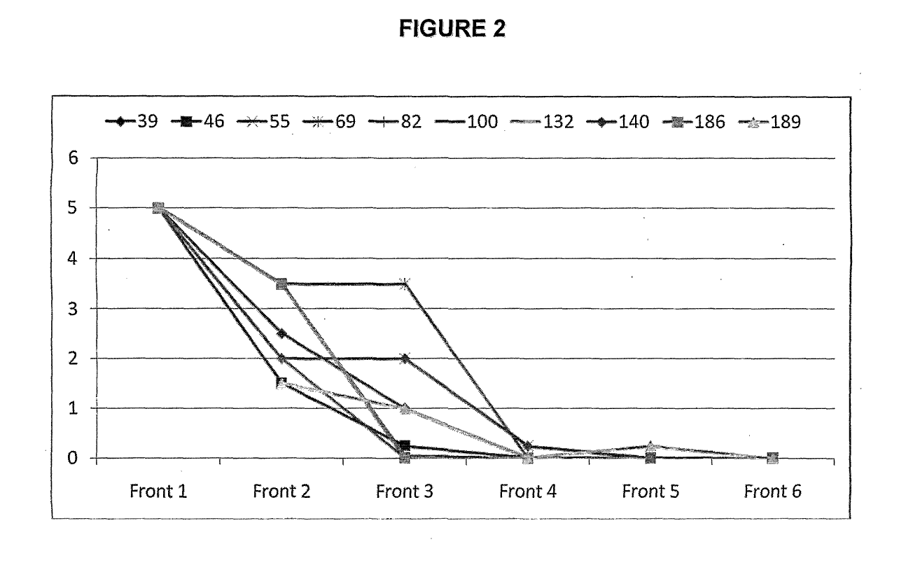

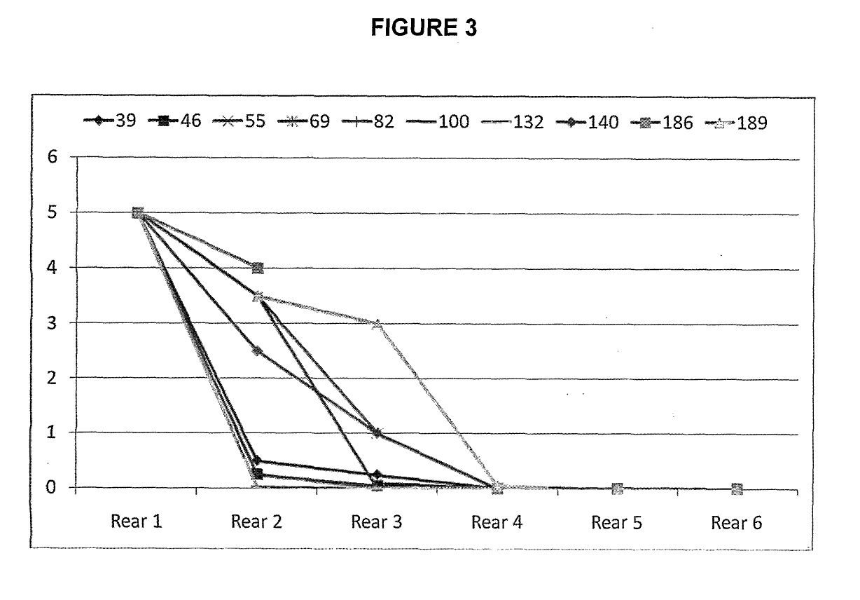

Changes in the scores among front or rear teats of the same cow at different time points post treatment are presented in FIGS. 2 and 3. The changes in the scores were somehow bigger in the rear quarters compared to the front quarters.

### Discussion

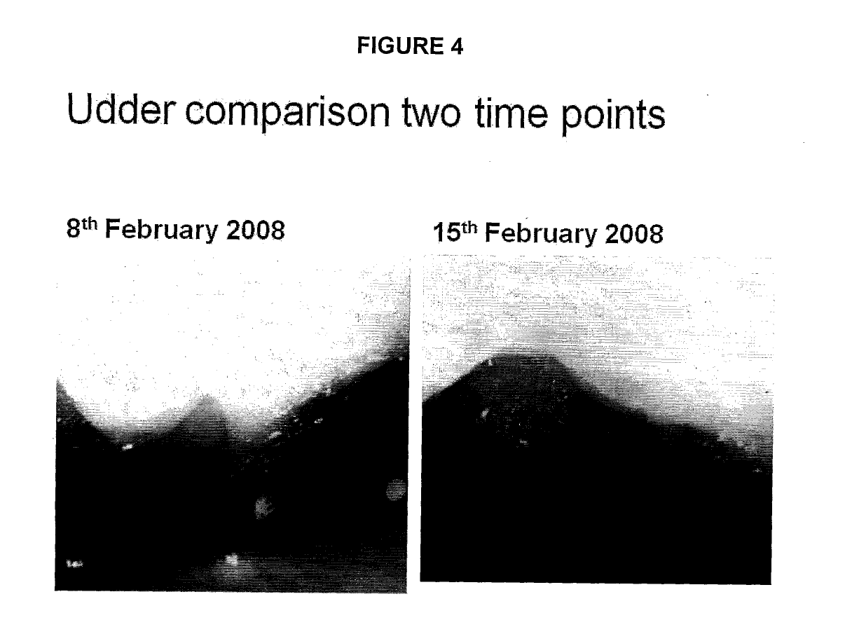

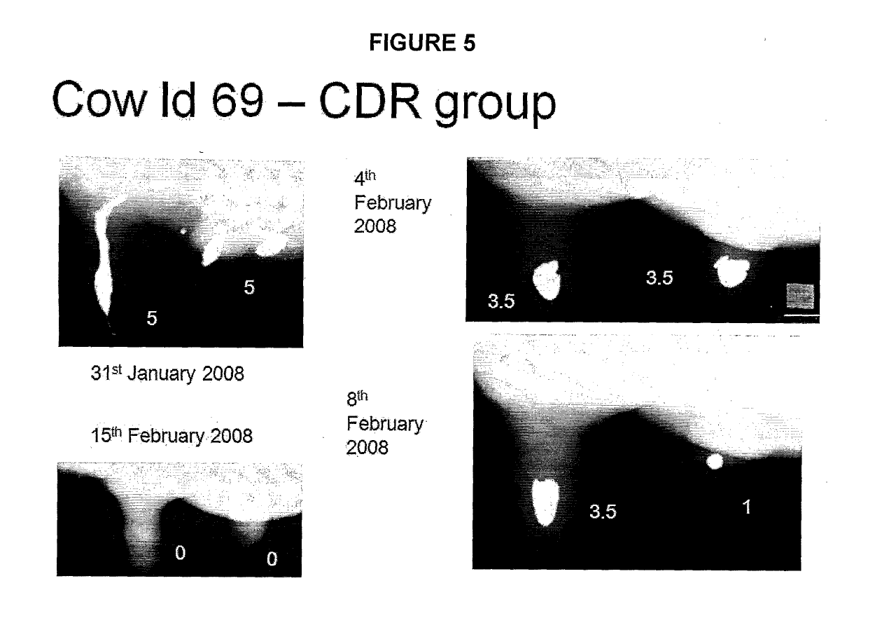

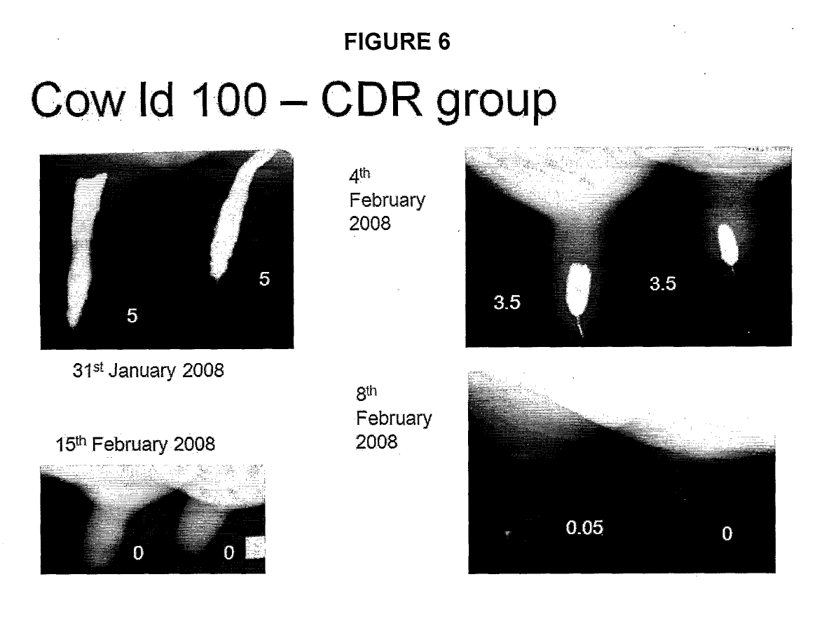

Preliminary analysis (not adjusted for missing values or cow/quarter effect) indicate the following:
1. Throughout the study, there was no evidence of leakage of the product or milk from the treated teats. There was one exception, where one teat of one cow was leaking milk. On the x-ray film, the droplet was clearly visible. It was hypothesised that the milk had higher density than normal milk, probably due to the barium content in the milk.
2. Nearly whole amount of the administered product dispersed in the udder during the first 15 days post treatment. The particles size in the udder was larger in the early stages of dispersion. Later, these particles become smaller (FIG. 4). The changes in the particles size was depressed during the steady stage of involution, when there was no fluid in the mammary glands.
3. The dispersion of the product was faster in the rear quarters. This likely occurred due to the higher milk production in the rear quarters. Consequently, a higher volume of dry secretion was formed and had to be absorbed. A second hypothesis is that the faster dispersion of the product in the rear quarters occurred due to better massage with the hind leg during the normal cows' movements.
4. The effective period of the product persistence in the teat cavity was less than a week. The highest susceptibility to the ascending infections is in the first week or maximum two post drying-off. During its persistence an artificial plus, similar to the Teatseal™ action was formed. Thus, it can be assumed that an effective barrier to the ascending infections was formed. This was confirmed by a challenge study, using barium-based teat sealant in early 2007. During the challenge study treated cow were twice challenged with a broth of Strep uberis at day 2 and 4 post drying-off. In that study, Barium-based ATS-Bomac performed better than the Teatseal™ in the prevention of new intramammary infections occurring in the early dry period.
5. The results of x-ray scores analysis confirmed that an early dispersion of the product occurred in the lower portions of the udder (FIGS. 5 and 6). It is likely this to occur throughout the gland tissue. For the DCT products to be effective early dispersion of the drug from the formulation is required. This should happen before the mammary secretion is too tick, in very small amounts (approximately end of second week of the dry period). The x-ray analysis suggests early dispersion of the product in the lower portion, and probably throughout the udder.

Aspects of the present invention have been described by way of example only and it should be appreciated that modifications and additions may be made thereto without departing from the scope thereof as defined in the appended claims.

What I/We claim is:

1. A formulation for administration to the teat canal of the mammary gland of an animal, the formulation including:
a physical barrier material,
characterised in that
the formulation includes sufficient antibiotic in relation to physical barrier material such that the formulation is configured to disintegrate over a period of time after administration to the teat canal.

2. The formulation as claimed in claim 1 wherein the formulation is a paste prior to administration.

3. The formulation as claimed in claim 1 or claim 2 wherein the formulation solidifies in the teat canal to form a substantially solid and coherent temporary physical barrier.

4. The formulation as claimed in any one of claims 1 to 3 wherein the physical barrier is of sufficient coherence to allow it to flex with the movement of the sides of the teat canal and/or lower portion of the teat cistern.

5. The formulation as claimed in any one of claims 1 to 4 wherein the physical barrier includes a barium based compound.

6. The formulation as claimed in claim 5 wherein the barium based compound is a barium salt.

7. The formulation as claimed in claim 6 wherein the barium salt is barium sulphate or barium substitute or combinations thereof.

8. The formulation as claimed in any one of claims 5 to 7 wherein the barium based compound or compounds is micronised.

9. The formulation as claimed in any one of claims 5 to 8 wherein the barium based compound or compounds is present in the formulation at a concentration of approximately 40% to 80% w/w.

10. The formulation as claimed in any one of claims 5 to 8 wherein the barium based compound or compounds is present in the formulation at a concentration of approximately 67% w/w.

11. The formulation as claimed in any one of claims 1 to 10 wherein the formulation includes a carrier.

12. The formulation as claimed in claim 11 wherein the carrier is a gelling compound or an oil or combination thereof.

13. The formulation as claimed in claim 12 wherein the gelling compound or compounds are present at a concentration within the range of approximately 1%-12% w/w of the oil component.

14. The formulation as claimed in claim 12 wherein the gelling compound or compounds are present at a concentration of approximately 5.5%-6% w/w of the oil compound.

15. The formulation as claimed in any one of claims 12 to 14 wherein the gelling compound is aluminium stearate.

16. The formulation as claimed in claim 15 wherein the aluminium stearate is present at a concentration of approximately 1.8% w/w of the formulation.

17. The formulation as claimed in any one of claims 12 to 16 wherein the oil is an oil that is difficult for the animal to metabolise.

18. The formulation as claimed in any one of claims 12 to 17 wherein the oil is non-absorbable.

19. The formulation as claimed in any one of claims 12 to 18 wherein the oil is present at a concentration of approximately 30% w/w.

20. The formulation as claimed in any one of claims 12 to 19 wherein the oil is liquid paraffin.

21. The formulation as claimed in any one of claims 12 to 20 wherein the ratio of barium based compound to oil is approximately between 60:35 to 70:25, respectively.

22. The formulation as claimed in claim 21 wherein the ratio between barium sulphate to oil is approximately 67:30, respectively.

23. The formulation as claimed in claim 21 wherein the ratio between Bi subnitrate to oil is approximately 65:31.

24. The formulation as claimed in any one of claims 12 to 23 wherein the preferred physical characteristics of the formulation are provided by the barium sulphate, along with at least one oil and at least one gelling agent.

25. The formulation as claimed in any of claims 1 to 24 which includes the thickening agent of Aerosil 200.

26. The formulation as claimed in any of claims 1 to 25 which includes the preservatives of methylparaben (as free acid) or propylparaben (as free acid) or combinations thereof.

27. The formulation as claimed in any one of claims 1 to 26 wherein the antibiotic is cephalosporin or functional equivalents thereof.

28. The formulation as claimed in any one of claims 1 to 26 wherein the antibiotic is penicillin or functional equivalents thereof.

29. The formulation as claimed in any one of claims 26 to 28 wherein the single antibiotic dosage is 0.2-2 grams per quarter.

30. The formulation as claimed in claim 27 wherein the cephalosporin dosage is between approximately 0.1-0.5 grams per quarter.

31. The formulation as claimed in claim 28 wherein the single penicillin dosage is approximately 0.1-1 gram per quarter.

32. The use, in the manufacture of a medicament, of the formulation as claimed in any one of claims 1 to 31, for the treatment, prevention or amelioration of mastitis in an animal.

33. The use of a formulation as claimed in claim 32 wherein the mammary gland is the whole of the udder of an animal, including the secreting tissue, connective tissue, glands and teats.

34. The use of a formulation as claimed in any one of claims 32 to 33 wherein the physical barrier disintegrates to leave minimal residue at calving.

35. The use of a formulation as claimed in any one of claims 32 to 34 wherein the physical barrier naturally deteriorates or degrades without the need for physical removal.

36. The use of a formulation as claimed in any one of claims 32 to 35 wherein the paste is administered through the teat canal.

37. The use of a formulation as claimed in any one of claims 32 to 36 wherein the paste is administered through the teat canal using a tube or syringe.

38. The use of a formulation as claimed in any one of claims 35 to 37 for treatment of dairy animals.

39. The use of a formulation as claimed in any one of claims 32 to 38 wherein the formulation is administered substantially at the start of the animal's dry period.

40. The use of a formulation as claimed in any one of claims 32 to 39 wherein the formulation is administered substantially during the drying off period.

41. The use of a formulation as claimed in any one of claims 32 to 39 wherein the formulation is administered substantially in the late pre-calving period prior to the first calving of priniparous heifers.

42. The use of a formulation as claimed in any one of claims 32 to 41 wherein the inclusion of an antibiotic in combination with a barium material that disintegrates over a predefined period time within the teat canal ensures that the antibiotic is introduced to the animal at the required dosage rate.

43. The use of a formulation as claimed in any one of claims 32 to 42 wherein the dissolution rate of the barium material is such that the appropriate dosage level of antibiotic is provided within the teat canal and cistern.

44. The use of a formulation as claimed in any one of claims 32 to 43 wherein the period of time in which the antibiotic is delivered is determined by the dissolution rate of the teat seal.

45. The use of a formulation as claimed in any one of claims 32 to 44 wherein the antibiotic is processed prior to the mandatory withholding period of eight milkings.

46. The use of a formulation as claimed in any one of claims 32 to 45 wherein the action of the antibiotic and teat seal is complimented by the production of a natural teat seal in the form of a keratin plug which forms when the artificial seal is in position.

47. The use of a formulation as claimed in any one of claims 32 to 46 wherein after the antibiotic has dissipated, the natural teat seal provides a physical barrier preventing further entry of micro organisms into the teat canal.

48. The use of a formulation as claimed in any one of claims 32 to 47 wherein the artificial teat seal dissolves over time, preventing any need to remove the teat seal from the teat canal.

49. The formulation substantially as herein described and illustrated by the examples and figures excluding the prior art disclosed within the specification.

50. The use of a formulation substantially as herein described and illustrated by the examples and figures excluding the prior art disclosed within the specification.
